(19) United States
(12) Patent Application Publication
(10) Pub. No.: US 2026/0335190 A1
(43) Pub. Date: Oct. 29, 2026

(54) System for Providing Ready-to-Use Acetylcholine Injection Solutions

(71) Applicants: Robert Bosch Gesellschaft für medizinische Forschung mbh, Stuttgart (DE); Robert Bosch GmbH, Stuttgart (DE)

(72) Inventors: Peter Ong, Stuttgart (DE); Thomas Muerdter, Weinstadt (DE)

(21) Appl. No.: 19/651,725

(22) Filed: Apr. 19, 2026

(30) Foreign Application Priority Data
Apr. 23, 2025 (DE) 10 2025 115 542.9

(51) Int. Cl. A61J 1/20; A61J 1/06; B65B 3/00

(52) U.S. Cl. CPC A61J 1/202; A61J 1/06; B65B 3/003

(57) ABSTRACT

A system for providing ready-to-use acetylcholine injection solutions with different acetylcholine concentrations is disclosed. The system includes at least five first receiving units. Each of the five first receiving units contains a quantity of acetylcholine or of an acetylcholine analog as a solid. The contained quantities of acetylcholine or of the acetylcholine analog differ among the five first receiving units, such that the quantity of acetylcholine or of the acetylcholine analog in four of the at least five first receiving units is greater in each subsequent first receiving unit than in the directly preceding first receiving unit. The quantity of acetylcholine or of the acetylcholine analog in the fifth of the at least five first receiving units is less than the highest quantity in the first four receiving units. The system is intended for application in the field of cardiovascular medicine, more specifically the intracoronary acetylcholine provocation test for detecting a CVD during cardiac catheterization.

This application claims priority under 35 U.S.C. § 119 to patent application no. DE 10 2025 115 542.9, filed on Apr. 23, 2025 in Germany, the disclosure of which is incorporated herein by reference in its entirety.

The present disclosure relates to a system for providing ready-to-use acetylcholine injection solutions, the use of the system in diagnostics, as well as methods for producing ready-to-use acetylcholine injection solutions, wherein the system is provided. Such systems, uses and methods are applicable in the field of cardiovascular diagnostics, and in particular for the intracoronary acetylcholine provocation test.

### BACKGROUND

Coronary vasomotor disorders (CVD) play a major role in the pathogenesis of angina pectoris and coronary heart disease. These diseases are characterized in that the tone of the vascular musculature of the coronary vessels is not correctly regulated to adjust the supply of blood and oxygen to the heart in accordance with demand. A central factor in controlling these processes is acetylcholine (ACh), a major neurotransmitter that is also responsible for regulating coronary vascular tone.

To diagnose CVD, an intracoronary provocation test with the administration of ACh can be performed. If a vasospasm of the coronary vessels is triggered by the administration of ACh, this is considered an indication of the presence of CVD. The test enables a functional assessment of coronary endothelial function and smooth vascular musculature and serves as an important tool for identifying patients with vasospastic angina or a coronary microcirculatory disorder.

To perform the intracoronary ACh provocation test, increasing doses of acetylcholine are administered into the coronary arteries via a cardiac catheter, starting, for example, at about 1.1×10⁻⁸ mol, until an effect occurs or the maximum dose of 1.1×10⁻⁶ mol is reached.

However, the ACh dosages (e.g., in ophthalmology) commonly used for other applications are too high for this test, so dilutions are required. These must be prepared individually and immediately prior to use, as ACh is unstable in aqueous solution. This results in significant time expenditure, potential dilution errors and a limited usable life of the solutions due to regulatory requirements.

Some studies have focused on the stability of ACh solutions at different concentrations, both high and low (Sletten et al., “Stability of acetylcholine chloride solution in autonomic testing”, J Neurol Sci. 234: 1-3 (2005)). New studies also show that low-concentration ACh solutions can remain stable under certain conditions (Neef et al., “Stability of Ready-To-Use-Solutions of Acetylcholine for Intracoronary Provocative Testing”, Research Letter, JACC: Basic to Translational Science 10(1) (2025)), which is relevant for practical application.

Furthermore, a pharmaceutical product for ophthalmological use described in DE 699 23 063 T2 offers a solution for the short-term preparation of ACh solutions, but is not optimized for intracoronary use.

The present disclosure aims to overcome the aforementioned disadvantages and facilitate implementation of the intracoronary ACh provocation test in clinical practice through improved methodology and composition. This is also advocated by the current guidelines of the European Society of Cardiology on chronic coronary syndrome (see also Vrints et al. European Heart Journal, Volume 45, Issue 36, Sep. 21, 2024, pages 3415-3537).

### SUMMARY

The object of the present disclosure is achieved by a system for providing ready-to-use acetylcholine injection solutions with different acetylcholine concentrations, wherein the system comprises: at least five first receiving units, wherein each of the five first receiving units contains a quantity (A; B; C; D; E) of acetylcholine or an acetylcholine analog as a solid, and wherein the contained quantities (A; B; C; D; E) of acetylcholine or the acetylcholine analog differ among the five first receiving units, such that the quantity (A; B; C; D) of acetylcholine or the acetylcholine analog in four of the at least five first receiving units is greater in each subsequent first receiving unit than in the directly preceding first receiving unit, and the quantity (E) of acetylcholine or the acetylcholine analog in the fifth of the at least five first receiving units is less than the highest quantity (D) in the four first receiving units.

The object is further achieved by the use of the system according to the disclosure in the diagnosis of coronary vasomotor disorders, preferably angina pectoris or coronary heart disease, in particular in an intracoronary acetylcholine provocation test, wherein, in particular, the quantities (A; B; C; D) of acetylcholine or of the acetylcholine analog are provided in the four first receiving units for the left coronary artery, and the quantity (E) of acetylcholine or the acetylcholine analog is provided in the fifth first receiving unit for the right coronary artery.

Finally, the object is achieved by a method for producing ready-to-use acetylcholine injection solutions with different acetylcholine concentrations, wherein the system according to the disclosure is used.

The system according to the disclosure advantageously allows the controlled and reproducible production of acetylcholine injection solutions with defined concentrations, thereby eliminating variability due to manual dilution processes. Since the quantities of acetylcholine or its analog are precisely specified and graduated in the first receiving units, the risk of dilution errors that may occur in manual preparation steps is significantly minimized. Furthermore, the direct availability of predefined acetylcholine concentrations reduces the time required to prepare the solutions, thereby allowing clinical and diagnostic procedures to be carried out more efficiently.

Also, by providing acetylcholine in solid form within the first receiving units, the chemical stability of the active ingredient is optimized, which prolongs the shelf life of the individual components in the system according to the disclosure and reduces the need for frequent re-preparations. Since no open dilution steps are required, the risk of microbial or chemical contamination during preparation of the injection solution is significantly reduced. The graduated quantities of acetylcholine or its analog within the first receiving units allows for flexible adjustment of the concentration to specific diagnostic or therapeutic requirements.

The system according to the disclosure is therefore suitable, among other things, for integration into automated dosing and injection systems, thereby ensuring precise and reproducible application in clinical or experimental environments.

Furthermore, by preparing and providing the acetylcholine solutions in a standardized manner, compliance with regulatory requirements regarding drug manufacture and use can be improved.

A further advantage is that no complex dilution steps are required with the system according to the disclosure. This reduces the risk of human error, which is particularly beneficial in time-critical clinical applications.

According to the disclosure, the system comprises at least five, preferably exactly 5, 6, 7, 8, 9, 10, or more first receiving units. In this context, “receiving unit” includes any container designed to receive and/or store acetylcholine or acetylcholine analogs as a solid. The at least 5 receiving units serve as a central element for providing acetylcholine or acetylcholine analogs within the system and ensure that the substance is available as needed and in defined quantities. In this respect, “at least five” means that the system comprises five or more such units to allow for flexible adjustment of the quantities of acetylcholine provided, depending on the particular application requirements. Each of the five first receiving units contains a quantity of acetylcholine or an acetylcholine analog as a solid. In the present case, as in the relevant field, an “acetylcholine analog” is understood to mean a chemical compound that is structurally or functionally similar to acetylcholine and is capable of exerting the same or a comparable physiological effect, in particular as a neurotransmitter. The contained quantities (A; B; C; D; E) of acetylcholine or the acetylcholine analog contained differ among the five first receiving units, such that the quantity (A; B; C; D) of acetylcholine or of the acetylcholine analog in at least four of the at least five first receiving units is greater in each subsequent first receiving unit than in the directly preceding first receiving unit, and the quantity (E) of acetylcholine or of the acetylcholine analog in the fifth of the at least five first receiving units is less than the highest quantity (D) in the four of the five first receiving units. Furthermore, “the quantity of acetylcholine or of the acetylcholine analog is greater in each subsequent first receiving unit than in the directly preceding first receiving unit” is intended to mean that the mass or concentration of the acetylcholine or of acetylcholine analog contained in a subsequent first receiving unit is at least one defined unit, such as, for example, micrograms or moles, above the quantity of the directly preceding first receiving unit, to ensure precise and scalable dosing.

In other words, for example, if five first receiving units are present in the system according to the disclosure, then with a quantity A in the first of the five first receiving units, a quantity B in the second of the five first receiving units, a quantity C in the third of the five first receiving units, a quantity D in the fourth of the five first receiving units, and a quantity E in the fifth of the five first receiving units: A<B<C<D, and E<D.

In particular in an intracoronary acetylcholine provocation test, preferably the quantities A, B, C, and D are provided for the left coronary artery and the quantity E for the right coronary artery.

According to one embodiment, the system according to the present disclosure comprises the at least 5 first receiving units described above with the stated quantities (A; B; C; D; E) of acetylcholine or of an acetylcholine analog as a solid, as well as a single container, for example an ampoule, comprising a dilution solution.

This embodiment has the advantage that a volume of the dilution solution can then be sequentially removed from the container and added to the receiving units to achieve the desired acetylcholine concentrations.

According to the disclosure, the acetylcholine or the acetylcholine analog is present as a solid material, preferably as a salt consisting of the acetylcholine cation and the anion of a suitable organic or inorganic acid, e.g., acetic acid, formic acid, tartaric acid, citric acid, gluconic acid, glutamic acid, carbonic acid, phosphoric acid, sulfuric acid, or hydrohalic acid, in particular as acetylcholine chloride/acetylcholine chloride analog or acetylcholine bromide/acetylcholine bromide analog.

Acetylcholine is (2-(trimethylammonium)ethyl) acetate and, as such, is a cation. The presence of acetylcholine as a solid, which is a salt, advantageously leads to increased stability of the acetylcholine, since it decomposes in aqueous environments. In the present case, it is particularly advantageous if the acetylcholine is present as a chloride, since chloride naturally occurs in the human body. Acetylcholine is also more stable in the form of salts, in particular and for example as acetylcholine chloride. This salt is a white crystalline powder that is commercially available.

The stability of acetylcholine is reduced, in particular at an acidic or basic pH. It is therefore advantageous if, in addition to the acetylcholine, pharmaceutically acceptable carriers and/or additives are also provided in the receiving units, which additives regulate or stabilize the pH value. In this way, the stability of the acetylcholine is increased after dissolution in water or an aqueous solution.

Preferred quantities or quantity ranges of acetylcholine or of the acetylcholine analog as a solid for the at least five receiving units are as follows: a quantity of acetylcholine or of an acetylcholine analog is contained in the first of four of the at least five first receiving units as a solid, wherein the quantity is measured such that, upon complete dissolution in a defined volume of a dilution solution, an acetylcholine/acetylcholine analog concentration of between 0.01 and 1.0 μg/mL, preferably between 0.05 and 0.8 μg/mL, even more preferably between 0.1 and 0.5 μg/mL, and further preferably 0.2 μg/mL is achieved in the ready-to-use injection solution, including the specifically stated limit values in each case; in the second of the four at least five first receiving units following the first, an acetylcholine/acetylcholine analog concentration of between 1.1 and 3.0 μg/mL, preferably between 1.3 and 2.7 μg/mL, even more preferably between 1.5 and 2.5 μg/mL, and further preferably 2.0 μg/mL is achieved, including the specifically stated limit values in each case; in the third of the four at least five first receiving units following the second, an acetylcholine/acetylcholine analog concentration of between 9.1 and 19.0 μg/mL, preferably between 9.5 and 15.0 μg/mL, even more preferably between 9.5 and 12.0 μg/mL, and further preferably 10 μg/mL is achieved, including the specifically stated limit values in each case; and in the fourth of four of the at least five first receiving units, an acetylcholine/acetylcholine analog concentration of between 19.1 and 30.0 μg/mL, preferably between 19.5 and 25.0 μg/mL, even more preferably between 19.5 and 23.0 μg/mL, and further preferably 20 μg/mL is achieved, including the specifically stated limit values in each case; and wherein in a fifth of the five first receiving units, the quantity of which is smaller than the highest quantity in the four first receiving units of the at least five first receiving units, an acetylcholine/acetylcholine analog concentration of between 3.1 and 9.0 μg/mL, preferably between 5.0 and 9.0 μg/mL, even more preferably between 6.0 and 8.5 μg/mL, and further preferably 8.0 μg/mL is present including the specifically stated limit values in each case.

By way of example, when ready-to-use 10 mL injection solutions are to be provided, the following quantities of acetylcholine or an acetylcholine analog are present as a solid in the first five receiving units: in the first of four of the at least five first receiving units, a quantity of between 0.1 μg and 10μg, preferably between 0.5 μg and 8 μg, even more preferably between 1.0 μg and 5.0 μg, and further preferably 2.0 μg, including the stated limit values in each case; in the second of four of the at least five first receiving units, a quantity of between 11 μg and 30 μg, preferably between 13 μg and 27 μg, even more preferably between 15 μg and 25 μg, and further preferably 20 μg, including the stated limit values in each case; in the third of four of the at least five first receiving units, a quantity of between 91 μg and 190 μg, preferably between 95 μg and 150 μg, even more preferably between 95 μg and 120 μg, and further preferably 100 μg, including the stated limit values in each case; and in the fourth of four of the at least five first receiving units, a quantity of between 191 μg and 300 μg, preferably between 195 μg and 250 μg, even more preferably between 195 μg and 230 μg, and further preferably 200 μg, including the stated limit values in each case; and wherein, in the fifth of the at least five first receiving units, the quantity of which is less than the highest quantity of the four of the first five receiving units, a quantity of between 31 μg and 90 μg, preferably between 50 μg and 90 μg, even more preferably between 60 μg and 85 μg, and further preferably 80 μg, is present, including the stated limit values in each case.

It will be appreciated that the above quantities are exemplary and can be individually adapted to the respective requirements, the patient, and the specific use, with the essential aspect being that the quantities in the first five receiving units are increasing.

According to a preferred embodiment, the system further comprises at least five second receiving units, wherein each of the five second receiving units contains a quantity of a dilution solution, and wherein a first receiving unit is associated with a second receiving unit in each case.

This embodiment has the advantage that, due to the direct association of each of the first receiving units with acetylcholine solid with a second receiving unit with a defined quantity of dilution solution, the preparation of a ready-to-use injection solution is facilitated, since the exact dilution is thus “automatically” ensured. Moreover, by combining different quantities of acetylcholine solid with graduated quantities of dilution solution, different concentrations can be produced as needed and quickly without additional manual calculations or work steps. Since each acetylcholine unit is already associated with its suitable dilution, the risk of dosing errors is minimized. This increases safety, which is particularly critical in critical applications such as injections. Furthermore, the direct association allows immediate preparation of the injection solutions, saving time and making the entire process more efficient.

Advantageously, the system can also be easily expanded by additional receiving units to provide an even greater range of concentrations without changing the basic structure.

The dilution solution is sterile; preferably it is selected from one of the following: saline solution (e.g., 0.45% or 0.9% (w/v) sodium chloride, NaCl), glucose solution (e.g., 5% or 10% (w/v) dextrose), water for injection (Aqua ad iniectabilia), Ringer's solution, Ringer's lactate solution (Hartmann's solution), sodium hydrogen carbonate solution.

General additional ingredients of the dilution solution may be citric acid, salts of citric acid, calcium salts, potassium salts, glycine or other amino acids, and other constituents suitable for the preparation of injection solutions.

According to a preferred embodiment of the system according to the disclosure, the association of each of the at least five first receiving units with one of the at least five second receiving units is performed by a labeling system, preferably consisting of colors, numbers, or symbols. Each first receiving unit has a label that matches the label of a corresponding second receiving unit, so that unique pairing is ensured. Overall, in the case of five first and five second receiving units, five different labels are provided in order to clearly distinguish the respectively associated receiving units from one another and to enable error-free association.

According to a further embodiment of the system according to the disclosure, a first receiving unit and a second receiving unit are respectively connected in a common container to form at least five common containers, with barrier means provided between a first receiving unit and a second receiving unit, which barrier means are at least partially removable.

By connecting a first and a second receiving unit in a common container, transport, storage, and provision are significantly facilitated, since fewer individual parts have to be handled. The provided, at least partially removable, barrier means further enable a targeted release of the dilution solution for mixing with the acetylcholine solid. This ensures that the solution is prepared only immediately prior to use, which increases stability. With the system according to the disclosure, the effort for manual mixing processes can be further reduced and quick, reproducible preparation of the injection solutions can be achieved. With this embodiment, mix-ups can also be ruled out and error-free use can be supported, since each common container contains exactly one pair of a first and a second receiving unit.

In a further preferred embodiment of the system according to the disclosure, the common container is a syringe or an ampoule, with at least partially removable barrier means provided between the at least five first receiving units and the at least five second receiving units.

In the present case, an “ampoule” is understood to mean a sealed container, typically made of glass or plastic, containing a liquid or a solid in a sterile environment. It is used for storage, transport and later removal of the contents, and is opened by breaking, cutting, or by a special opening device. A “syringe” is understood to mean a medical instrument consisting of a cylindrical hollow body (cylinder) with a movable plunger, which is used to draw up, dose, and inject liquids. It is typically provided with a needle or a connector to precisely draw up or administer liquids.

The use of a syringe or ampoule as a common container offers the advantage that proven, easy-to-handle solutions can be used. The at least partially removable barrier means allow uncomplicated release of the dilution solution into the area with the acetylcholine solid, which allows the ready-to-use injection solution to be produced immediately within a single container.

Furthermore, the risk of material losses that could arise during transfer is minimized, since dilution and administration are carried out in the same container. Preparing the solution directly in a sterile syringe or ampoule also significantly reduces the risk of contamination, since no additional transfer processes are necessary. Finally, the immediate availability of the ready-to-use injection solution in an administration container significantly reduces the preparation time, which can be crucial, especially in emergency situations.

In the present case, “removable barrier means” is understood to mean any device or structure that at least partially prevents mixing or contact between two or more components within a container until a targeted removal of this barrier occurs. Barrier means may be mechanical or physical in nature and are designed to be removed, broken, or bypassed by manual, mechanical, or automatic operation to allow mixing of the separate components.

According to preferred embodiments of the system according to the disclosure, the barrier means may be selected from membrane seals, i.e., thin membranes that can be pierced by pressure, rotation, or a plunger stroke to allow mixing of the dilution solution and acetylcholine; frangible, i.e., breakable, connections, in particular fragile partitions or predetermined breaking points that can be selectively opened by mechanical stress, for example by bending or pressing; septa with a puncture device, in particular elastic rubber septa that are pierced with a needle; screw or rotation mechanisms, in particular rotatable closures or plungers that, when twisted, for example open a channel between the two receiving units; sliding seals or plunger valves, in particular movable seals that can be opened by a plunger or slider to mix the contents of two receiving units; foil seals with a peel-off mechanism, i.e., foils that can be removed by peeling off or tearing; valve systems, for example one-way valves or check valves that, when actuated, allow liquid flow between the receiving units but prevent premature mixing.

According to a further preferred embodiment of the system according to the disclosure, the at least five common containers are structurally coupled, with separate first receiving units and second receiving units.

Advantageously, the structural coupling of the at least five common containers allows for a space-saving arrangement, which facilitates storage, transport, and handling. In addition, several units can be prepared, transported, or administered simultaneously by way of the coupling, without the need to remove or manage individual containers separately. Thus, all necessary concentrations are also available in a structurally connected system, thereby allowing a quick change between the desired concentrations. The structural coupling also ensures uniform storage and protects the individual receiving units from physical effects that could lead to damage or contamination. Despite the coupling, the first and second receiving units remain separate, so that individual units can be replaced or supplemented as needed without replacing the entire system. The structured arrangement of the containers facilitates systematic labeling and association, which prevents errors when selecting the desired concentration.

According to a preferred embodiment of the system according to the present disclosure, the barrier means can be removed between the at least five first receiving units and the at least five second receiving units simultaneously or independently of one another. By allowing the barrier means to be removed independently of one another, the user can selectively prepare a single injection solution without affecting the other receiving units.

On the other hand, the ability to remove all barrier means simultaneously makes it possible to provide several ready-to-use solutions at the same time, which can be particularly advantageous in time-critical situations, e.g., in the medical field.

Users can either prepare a single solution as needed or multiple solutions in parallel, without additional effort or waiting times.

In a further preferred embodiment of the system according to the disclosure, which comprises at least five second receiving units in addition to the five first receiving units, and each of the five second receiving units contains a quantity of a dilution solution, and wherein each first receiving unit is respectively associated with a second receiving unit, the five first receiving units are structurally separated from the at least five second receiving units.

This embodiment offers the advantage of allowing the user to flexibly decide what quantities or concentrations are needed, as each unit is individually accessible. The system can also be easily expanded or adjusted by adding or removing individual receiving units without compromising the integrity of the remaining units.

In this embodiment, for example, the second receiving units can be associated with a respective first receiving unit via the labeling systems described above, for example, colors, numbers, symbols, or other markings.

In preferred embodiments, the at least five first receiving units are each ampoules, i.e., the five first receiving units are contained in ampoules, and the at least five second receiving units are each syringes, i.e., the five second receiving units are contained in syringes, or the at least five first receiving units are each syringes, or contained therein, and the at least five second receiving units are each ampoules, or contained therein.

The selectable combination of either syringes as the first receiving units and ampoules as the second or vice versa allows the system to be adapted to various user needs and preferences.

Syringes as the first receiving units allow for immediate and precise dosing of the acetylcholine solid, while ampoules as the second receiving units ensure a sterile and secure provision of the dilution solution—or vice versa, depending on the preferred handling. In addition, using standard syringes and ampoules can facilitate everyday clinical use. The combination of syringe and ampoule—in both variants—allows the ready-to-use injection solution to be prepared quickly and precisely by simple mechanical processes (e.g., drawing up or injecting the dilution solution). This system can also be scaled as required, as both ampoules and syringes are available in different volumes and configurations.

According to a further embodiment of the system according to the disclosure, dilution solutions can each be supplied to the at least five first receiving units via an automatic or semi-automatic supply system.

An automatic or semi-automatic supply system ensures accurate dosing of the dilution solution, minimizing human errors in preparation. The automated supply may further significantly reduce manual effort and allow for rapid provision of ready-to-use injection solutions, especially when preparing multiple units simultaneously.

The standardized supply ensures that each injection solution is prepared in identical concentration and quality, which increases the reproducibility and reliability of the system. The risk of contamination from manual handling can also be significantly reduced. Furthermore, by way of programmable or adjustable parameters in the supply system, different quantities of dilution solution may be supplied to produce different concentrations flexibly and without additional effort.

The system according to the disclosure may further include application information that is provided either on each of the five first and five second receiving units, or on a common container for a respective first and a second receiving unit, as described above, and/or specified in package insert materials for the system and/or provided electronically, preferably via the Internet.

When the application information is applied to each individual vessel, the vessel may be glass or plastic and printed with application information.

If the application information is specified in package insert materials for the system, the package insert materials may be paper printed with application information.

If the application information is provided electronically, the application information may be provided in a publicly accessible area of the Internet or in an area accessible only upon identification, optionally personalized.

As mentioned above, the present disclosure also relates to the use of the system according to the disclosure described above in all of its embodiments in the diagnosis of coronary vasomotor disorders, preferably angina pectoris or coronary heart disease, in particular in an intracoronary acetylcholine provocation test, in particular in an intracoronary acetylcholine provocation test for detection of CVD during cardiac catheterization, wherein, in particular, the quantities (A; B; C; D) of acetylcholine or of the acetylcholine analog are provided in four of the at least five first receiving units for the left coronary artery, and the quantity (E) of acetylcholine or of the acetylcholine analog is provided in the fifth first receiving unit for the right coronary artery.

Advantageously, and as mentioned above, the system according to the present disclosure enables the accurate and standardized provision of acetylcholine injection solutions, which is critical for reliable and reproducible results in the intracoronary acetylcholine provocation test. By providing different concentrations, the system can be customized to the patient to test different coronary artery reactions and allow for differentiated diagnostics. The system also ensures quick preparation of ready-to-use solutions directly in the clinical setting, which is particularly advantageous in time-critical diagnostic methods such as the provocation test. Standardized provision and optional automated supply systems reduce the risk of human error and ensure high sterility throughout the process.

Easy handling and rapid availability of different concentrations save time and resources in daily clinical practice and facilitate diagnostic procedures. In addition to the intracoronary acetylcholine provocation test, the system can also be used in other diagnostic and therapeutic applications, underscoring its versatility.

In particular, the system according to the disclosure and the associated acetylcholine test can also be carried out through bypass vessels, i.e., in particular also in patients who have previously undergone coronary artery bypass graft surgery (CABG) prior to the acetylcholine test, wherein the above-described dosages or quantities (A; B; C; D) (of acetylcholine or of the acetylcholine analog) are used in the first four receiving units provided for the left coronary artery.

As further mentioned above, the present disclosure also relates to a method for producing ready-to-use acetylcholine injection solutions with different acetylcholine concentrations, wherein the method comprises the steps of:
a) providing a system according to the disclosure in any of the embodiments described above; and
b) adding a defined quantity of a dilution solution to each of the at least five first receiving units.

In particular, it is preferred when step b) is carried out automatically or semi-automatically.

Furthermore, the present disclosure also relates to a method for producing ready-to-use acetylcholine injection solutions with different acetylcholine concentrations, wherein the method comprises the steps of:
a) providing a system according to the disclosure; and
b) adding a defined quantity of a dilution solution to each of the at least five first receiving units from at least five second receiving units, which are each associated with the at least five first receiving units.

In this embodiment of the method according to the disclosure, for example, the above-described ampoule-and-syringe combination system can be used, whereby the advantages described for this system also apply to the method, in which it is used.

Finally, the present disclosure also relates to a method for producing ready-to-use acetylcholine injection solutions with different acetylcholine concentrations, wherein the method comprises the steps of:
a) providing a system according to the disclosure in any of the embodiments described above;
b) at least partially removing the barrier means provided in each case between a first receiving unit and a second receiving unit; and
c) adding a defined quantity of a dilution solution to each of the at least five first receiving units from at least five second receiving units, which are each associated with the at least five first receiving units.

In the methods described above, by adding the dilution solutions, the acetylcholine quantities present in the first receiving units are each dissolved as the last step, as a result of which the ready-to-use acetylcholine injection solutions are prepared.

In this embodiment of the method according to the disclosure, one of the embodiments of the system according to the disclosure described above is employed, in which the five first and the five second receiving units are separated from one another by barrier means. The advantages described for the respective embodiments of this system thus also result for the method, in which they are used.

Further features, advantages and details of the present disclosure are evident from the attached figures and the subsequent description. It will be appreciated that the embodiments presented and described are exemplary only and the disclosure is not limited thereto.

### BRIEF DESCRIPTION OF THE DRAWINGS

FIG. 1 shows a schematic, not-to-scale illustration of a first exemplary embodiment of the system according to the disclosure, with 5 rows of two receiving units each, i.e., first receiving units and associated second receiving units, separated from each other by at least partially removable barrier means);

FIG. 2 shows a schematic, not-to-scale illustration of a second exemplary embodiment of the system according to the disclosure, with 5 syringes and 5 associated ampoules;

FIG. 3 shows a schematic, not-to-scale illustration of a third exemplary embodiment of the system according to the disclosure, with 5 syringes each comprising two receiving units, which are separated from each other by at least partially removable barrier means;

FIG. 4 shows a schematic, not-to-scale illustration of a fourth exemplary embodiment of the system according to the disclosure, in which dilution solutions are automatically or semi-automatically supplied to the first receiving units.

### DETAILED DESCRIPTION

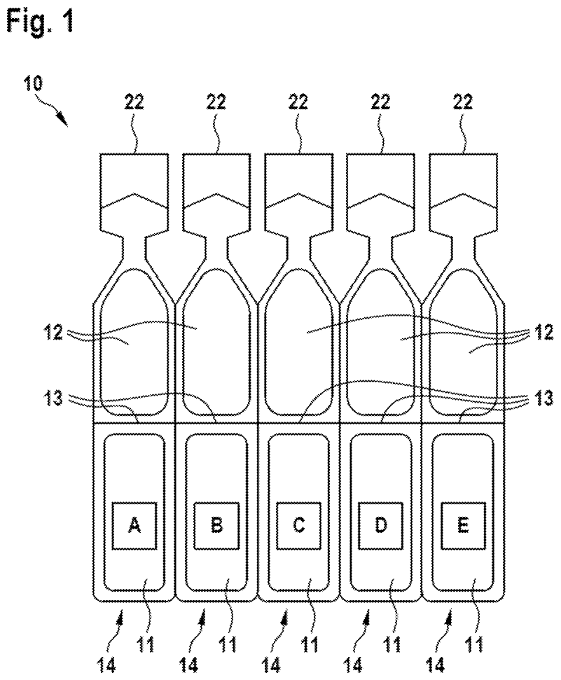

FIG. 1 shows a schematic, not-to-scale illustration of a first exemplary embodiment of the system 10 according to the disclosure. The system shown here by way of example comprises 5 rows, each with a first receiving unit 11 and an associated second receiving unit 12. The respective associated receiving units 11 and 12 are separated from each other by at least partially removable barrier means 13. The barrier means in the system 10 shown in FIG. 1, for example, represent a predetermined breaking point between each of the first and second receiving units 11 and 12.

A first receiving unit 11 and a second receiving unit 12 are each connected in a common container 14, here an ampoule 22. By way of example, as shown in FIG. 1, the containers 14 are structurally coupled, with separate first receiving units 11 and second receiving units 12, each of which are separated from each other by removable barrier means 13.

In the first receiving units 11, different quantities A, B, C, D, and E of acetylcholine are contained as a solid, wherein the acetylcholine quantity B, which is located in the receiving unit 11 following the receiving unit 11 containing acetylcholine quantity A, is greater than quantity A; furthermore, the acetylcholine quantity C, which is located in the receiving unit 11 following the receiving unit 11 containing acetylcholine quantity B, is greater than quantity B; furthermore, the acetylcholine quantity D, which is located in the receiving unit 11 following the receiving unit 11 containing acetylcholine quantity C, is greater than quantity C; and finally, the acetylcholine quantity E, which is located in the receiving unit 11 following the receiving unit 11 containing acetylcholine quantity D, is smaller than quantity D. In other words, A<B<C<D and E<D, which means that the acetylcholine quantity in four of at least 5 first receiving units is greater in each subsequent receiving unit 11 than in the directly preceding receiving unit 11.

The five second receiving units 12 contain quantities of dilution solutions a, b, c, d, and e that are identical in the example shown in FIG. 1. Upon removal of the barrier means 13, the dilution solutions a, b, c, d, and e provided in the five second receiving units 12 can mix with the respective acetylcholine quantities A, B, C, D, and E present in the respective associated receiving units 11, so that the respective acetylcholine dissolves and the ready-to-use acetylcholine injection solutions can be provided. These can, for example, each be drawn up with a syringe (not shown) and used for the desired purpose, in particular for an intracoronary acetylcholine provocation test.

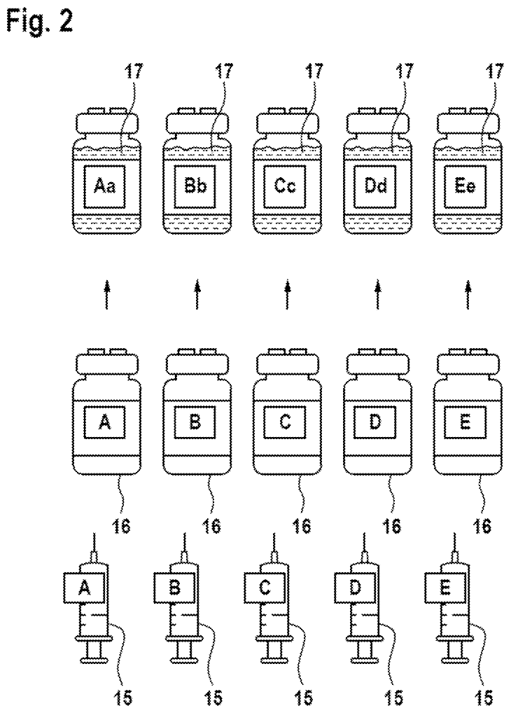

FIG. 2 shows a schematic, not-to-scale illustration of a second exemplary embodiment of the system 10 according to the disclosure. The system shown in FIG. 2, by way of example, includes five syringes 15 shown in the lower part of the figure and five associated ampoules 16 shown in the middle of the figure. The five ampoules 16 each form or contain one of the five first receiving units 11, in which the different quantities A, B, C, D, and E of acetylcholine are present as a solid, wherein the following also applies here: A<B<C<D, and E<D.

The syringes 15 shown in the lower part of FIG. 2 each form or contain one of the five second receiving units 12, in which dilution solutions a, b, c, d, e are present, the quantities of which are identical in the embodiment shown herein as an example. In the ampoules 16, the receiving units 11 are provided, and/or the ampoules 16 form the receiving units 11. In these, different acetylcholine quantities A, B, C, D, E are provided, wherein the following also applies here: A<B<C<D and E<D. The ampoules comprise a partially removable barrier means 13 in the form of a puncturable septum (not shown). A syringe 15 is respectively associated with an ampoule 16, and after puncturing, the dilution solution a, b, c, d, e can be mixed with the respectively associated acetylcholine quantities A, B, C, D, and E, whereby the acetylcholine can dissolve, and the finished injection solutions 17 (also referred to as Aa, Bb, Cc, Dd, Ee to illustrate the different concentrations) are provided, which contain different acetylcholine concentrations. The finished injection solutions 17 can then be removed from the ampoules 16, e.g., by way of a syringe (not shown), and used for the desired purpose, in particular for an intracoronary acetylcholine provocation test.

It will be appreciated that, conversely, the different quantities of acetylcholine in the receiving units 11 may be present in syringes, and the dilution solutions in the receiving units 12 may be present in ampoules.

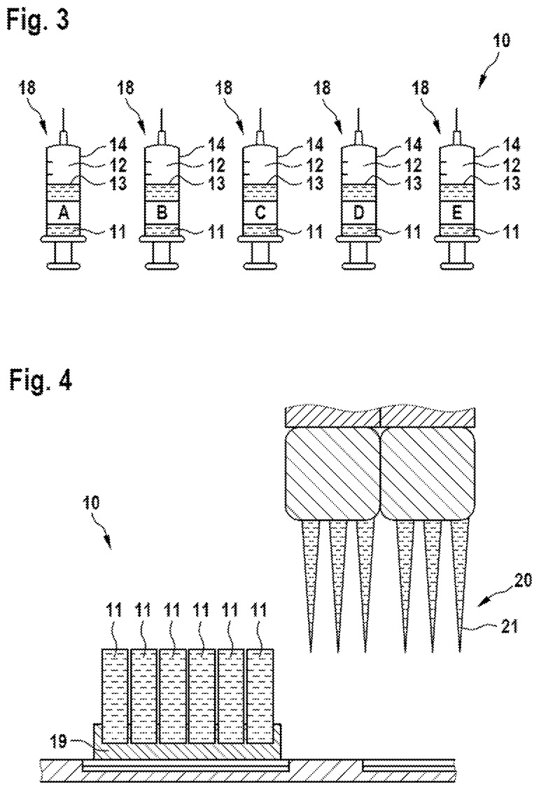

FIG. 3 shows a schematic, not-to-scale illustration of a third exemplary embodiment of the system 10 according to the disclosure. Five containers 14 in the form of syringes 18 are shown here, each comprising a first receiving unit 11 and an associated second receiving unit 12. The respective associated receiving units 11 and 12 are separated from each other by at least partially removable barrier means 13. The barrier means in the system 10 shown in FIG. 3 are, for example, membranes or septa, which may be broken or punctured to remove the barrier.

Likewise in this case, analogously to the exemplary embodiment shown in FIG. 1, a first receiving unit 11 and a second receiving unit 12 are connected in a common container 14, here a syringe. Unlike in the embodiment shown in FIG. 1, however, the containers 14 are not structurally coupled but are separated from each other.

Different quantities A, B, C, D, E of acetylcholine (as a solid) are contained in the first receiving units 11, wherein the following also applies to the acetylcholine quantities here—analogously to FIG. 1: A<B<C<D and E<D, which means that the acetylcholine quantity is greater in four of at least five subsequent receiving units 11 than in the directly preceding receiving unit 11.

The five second receiving units 12 contain quantities of dilution solutions a, b, c, d, and e that are identical in the example shown in FIG. 3. Upon removal of the barrier means 13, the dilution solutions a, b, c, d, and e provided in the five second receiving units 12 can mix with the respective acetylcholine quantities A, B, C, D, and E present in the respective associated receiving units 11, so that the respective acetylcholine dissolves and the ready-to-use acetylcholine injection solutions can be provided. These can be used directly in the syringe for the desired purpose, in particular for an intracoronary acetylcholine provocation test.

FIG. 4 shows a schematic, not-to-scale illustration of a fourth exemplary embodiment of the system 10 according to the disclosure. In this case, six first receiving units 11 are provided (wherein five or more than six may also be provided) by way of example, which may be, for example, ampoules or vials that are provided on a rack 19. The rack 19 serves as a holder, in which a plurality of receiving units is fixed in a defined arrangement. These six first receiving units 11 comprise acetylcholine as a solid in different quantities A, B, C, D, E, F.

For preparing the ready-to-use acetylcholine injection solutions, dilution solutions are subsequently pipetted in an automated manner into the receiving units 11, e.g., via a pipetting unit 20. Each first receiving unit 11 is identified by an identification feature (such as a color, number, symbol, or code), such that an automated system (not shown) recognizes which container is intended for which dilution step or pipetting operation. An automated pipetting unit then precisely moves pipetting needles 21 to the respective receiving units 11. The needle may then supply the dilution solution to prepare a ready-to-use acetylcholine injection solution with different acetylcholine concentrations.

Such a system 10 may be either fully automated or partially automated. In both cases, it ensures that the required quantity of dilution solution is pipetted into the corresponding receiving units 11 to achieve the desired concentrations.

What is claimed is:

1. A system for providing ready-to-use acetylcholine injection solutions with different acetylcholine concentrations, the system comprising:
at least five first receiving units, wherein each of the five first receiving units contains a quantity of acetylcholine or of an acetylcholine analog as a solid, and wherein the contained quantities of acetylcholine or of the acetylcholine analog differ among the five first receiving units, such that the quantity of acetylcholine or of the acetylcholine analog in four of the at least five first receiving units is greater in each subsequent first receiving unit than in the directly preceding first receiving unit, and the quantity of acetylcholine or of the acetylcholine analog in the fifth of the at least five first receiving units is less than the highest quantity in the four first receiving units.

2. The system according to claim 1, further comprising:
at least five second receiving units, wherein each of the five second receiving units contains a quantity of a dilution solution, and wherein a first receiving unit is associated with a second receiving unit in each case.

3. The system according to claim 2, wherein a first receiving unit and a second receiving unit are respectively connected in a common container to form at least five common containers, with an at least partially removable barrier provided between a first receiving unit and a second receiving unit.

4. The system according to claim 3, wherein the common container is a syringe or an ampoule, with the at least partially removable barrier provided between the at least five first receiving units and the at least five second receiving units.

5. The system according to claim 3, wherein the at least five common containers are structurally coupled, with separate first receiving units and second receiving units.

6. The system according to claim 3, wherein the barrier between the at least five first receiving units and the at least five second receiving units are removable simultaneously or independently of one another.

7. The system according to claim 2, wherein the at least five first receiving units are structurally separated from the at least five second receiving units.

8. The system according to claim 7, wherein the at least five first receiving units are each ampoules and the at least five second receiving units are each syringes, or wherein the at least five first receiving units are each syringes and the at least five second receiving units are each ampoules.

9. The system according to claim 1, wherein dilution solutions are supplied to each of the at least five first receiving units via an automatic or semi-automatic supply system.

10. A method of using the system according to claim 1 in the diagnosis of coronary vasomotor disorders in an intracoronary acetylcholine provocation test, wherein the quantities of acetylcholine or of the acetylcholine analog are provided in the first four receiving units for the left coronary artery, and the quantity of acetylcholine or of the acetylcholine analog is provided in the fifth receiving unit for the right coronary artery.

11. A method for producing ready-to-use acetylcholine injection solutions with different acetylcholine concentrations, the method comprising:
a) providing the system according to claim 1; and
b) adding a defined quantity of a dilution solution to each of the at least five first receiving units.

12. The method according to claim 11, wherein step b) is automated or semi-automated.

13. A method for producing ready-to-use acetylcholine injection solutions with different acetylcholine concentrations, the method comprising:
a) providing the system according to claim 2; and
b) adding a defined quantity of a dilution solution to each of the at least five first receiving units from at least five second receiving units, which are each associated with one of the at least five first receiving units.

14. A method for producing ready-to-use acetylcholine injection solutions with different acetylcholine concentrations, the method comprising:
a) providing the system according to claim 3;
b) at least partially removing the barrier provided in each case between a first receiving unit and a second receiving unit; and
c) adding a defined quantity of a dilution solution to each of the at least five first receiving units from at least five second receiving units, which are each associated with one of the at least five first receiving units.

15. The system according to claim 10, wherein the coronary vasomotor disorders are angina pectoris or coronary heart disease.
